(19) United States
(12) Patent Application Publication
(10) Pub. No.: US 2025/0198910 A1
(43) Pub. Date: Jun. 19, 2025

(54) METHODS AND SYSTEMS FOR CORRECTING BIAS

(71) Applicant: DH Technologies Development Pte. Ltd., SINGAPORE (SG)

(72) Inventor: John W. Silzel, Brea, CA (US)

(21) Appl. No.: 18/981,309

(22) Filed: Dec. 13, 2024

Related U.S. Application Data
(60) Provisional application No. 63/610,918, filed on Dec. 15, 2023.

(51) Int. Cl. G01N 21/25; G01N 1/40; G01N 27/447; G01N 33/68

(52) U.S. Cl. CPC G01N 21/25; G01N 1/40; G01N 27/44726; G01N 27/44782; G01N 33/6803; G01N 2001/4038

(57) ABSTRACT

A method for correcting detection bias includes detecting spectral data of a standard sample, the standard sample comprising two or more analytes. Each analyte has a known quantity, and the spectral data of the standard sample includes a peak for each of the two or more analytes. The method further includes determining a bias parameter for each of the two or more analytes based on the peak for each of the two or more analytes of the standard sample.

### CROSS-REFERENCE TO RELATED APPLICATION

This application claims priority to U.S. Provisional Application No. 63/610,918 filed Dec. 15, 2023, and entitled “METHODS AND SYSTEM FOR CORRECTING FLUORESCENT LABELING BIAS,” the disclosure of which is hereby incorporated by reference in its entirety.

### BACKGROUND

Analytes (e.g., DNA plasmids and proteins), may be labeled (e.g., using a fluorescent dye) to facilitate detection during their analysis (e.g., using separation by capillary electrophoresis). The amount of emitted or absorbed light per labeled analyte molecule (e.g., molecule of DNA or protein) might vary—e.g., it might depend on the number of dyes (e.g., fluorescent dyes) or their moieties intercalating or being otherwise associated with the analyte, the nature of the molecular environment immediately surrounding the label or dye, and other factors. This variation may result from, e.g., the dye concentration used during labeling, varying affinity of the dye for various analytes, varying affinity of the dye for different conformations of analytes, other components present during labeling (e.g., components of the solution in which the labeling is performed), or materials used for the separation and/or detection (e.g., composition of the solution, gel, etc. in which analytes are separated and/or detected). Thus, peak parameters that are measured for two different analytes (e.g., their peak areas) might not be proportional to the actual quantity (e.g., concentration) of those analytes in the sample. Thus, a need exists for detecting and/or correcting for this potential detection bias.

### SUMMARY

In one aspect, the technology relates to a method for correcting detection bias, the method including detecting spectral data of a standard sample, the standard sample including two or more analytes, each having a known quantity, the spectral data of the standard sample including a peak for each of the two or more analytes, and determining a bias parameter for each of the two or more analytes based on the peak for each of the two or more analytes of the standard sample.

In another example of the above aspect, the method further includes detecting spectral data of a sample of interest including the two or more analytes, the spectral data of the sample of interest including a peak for each of the two or more analytes, and determining one or more quantities of the two or more analytes in the sample of interest using the bias parameters for each of the two or more analytes. In another example, determining the bias parameter for each the two or more analytes includes determining a peak area for each of the two or more analytes in the spectral data of the standard sample. In a further example, determining the bias parameters is performed by using equation Ci=Aᵢfᵢ/(Σₖ₌₁ⁿ Aₖfₖ), where n is a total number of the two or more analytes in the standard sample, i is an analyte index from 1 to n, Cᵢ is the known quantity of an i^{th} analyte in the standard sample, Aᵢ is the peak area for the i^{th} analyte in the spectral data of the standard sample, and fᵢ is the bias parameter for the i^{th} analyte.

In another example of the above aspect, the two or more analytes include a supercoiled (S), a linear (L), and a nicked-open circular (N) plasmids, and determining the bias parameters is performed by using equations:

$$\begin{matrix}{A}_{S} {f}_{S} = {C}_{S} ( {A}_{S} {f}_{S} + {A}_{N} {f}_{N} + {A}_{L} {f}_{L} ) \\ {A}_{N} {f}_{N} = {C}_{N} ( {A}_{S} {f}_{S} + {A}_{N} {f}_{N} + {A}_{L} {f}_{L} ) \\ {A}_{L} {f}_{L} = {C}_{L} ( {A}_{S} {f}_{S} + {A}_{N} {f}_{N} + {A}_{L} {f}_{L} )\end{matrix}$$

A_{S}, A_{N}, and A_{L} are the peak areas for the supercoiled (S), nicked-open circular (N), and linear (L) plasmids in the spectral data of the standard sample, C_{S}, C_{N}, and C_{L} are the known quantities for the supercoiled (S), nicked/open circular (N), and linear (L) plasmids in the standard sample, and f_{S}, f_{N}, and f_{L} are the bias parameters for the supercoiled (S), nicked/open circular (N), and linear (L) plasmids.

In a further example, determining the bias parameter for each the two or more analytes includes determining an eigenvector (f) of a matrix having a formula A⁻¹ca, wherein a is a row vector of the peak areas for the two or more analytes, c is a column vector of the known quantities of the two or more analytes of the standard sample, A is a diagonal matrix with the peak areas for the two or more analytes along the main diagonal, and wherein f is a column vector of the bias parameters for the two or more analytes. In yet another example, the two or more analytes include a supercoiled (S), a linear (L), and a nicked/open circular (N) plasmids, and

$$\begin{matrix}a = ( {A}_{S} {A}_{N} {A}_{L} ) \\ c = ( \begin{matrix}{C}_{S} \\ {C}_{N} \\ {C}_{L}\end{matrix} ) \\ {A}^{- 1} = ( \begin{matrix}1 / {A}_{S} & 0 & 0 \\ 0 & 1 / {A}_{N} & 0 \\ 0 & 0 & 1 / {A}_{L}\end{matrix} ) and \\ f = ( \begin{matrix}{f}_{S} \\ {f}_{N} \\ {f}_{L}\end{matrix} )\end{matrix}$$

A_{S}, A_{N}, and A_{L} are the peak areas for the supercoiled (S), nicked/open circular (N), and linear (L) plasmids, C_{S}, C_{N}, and C_{L} are the known quantities for the supercoiled (S), nicked/open circular (N), and linear (L) plasmids, and f_{S}, f_{N}, and f_{L} are the bias parameters for the supercoiled (S), nicked/open circular (N), and linear (L) plasmids.

In yet another example, the two or more analytes include one of a protein and a nucleic acid. In a further example, the two or more analytes include a supercoiled (S) plasmid, a linear (L) plasmid, and/or a nicked/open circular (N) plasmid.

In other examples, the standard sample is subjected to capillary electrophoresis separation in a separation matrix prior to detecting the spectral data. In a further example, the standard sample is labeled with a dye prior to detecting the spectral data. In other example, the method further includes determining the bias parameters for each of the two or more analytes under two or more different conditions to determine under which of the two or more different conditions the bias parameters are closer to 1.

In additional examples, the two or more different conditions include at least one of (a) different separation matrices used for capillary electrophoresis separation of the standard sample, and (b) different dyes used for labeling the standard sample. For example, determining the one or more quantities of the two or more analytes in the sample of interest using the bias parameters for each of the two or more analytes includes using equation ^{U}Cᵢ=^{U}Aᵢfᵢ/(Σₖ₌₁^{n U}Aₖfₖ), where n is a total number of the two or more analytes in the sample of interest, i is an analyte index from 1 to n, ^{U}Cᵢ is the quantity of an i^{th} analyte in the sample of interest, ^{U}Aᵢ is a peak area for the i^{th} analyte in the spectral data of the sample of interest, and fᵢ is the bias parameter for the i^{th} analyte.

In another aspect, the technology relates to a computer-implemented method for correcting detection bias including receiving, using a processor, a first data set including an spectral data of a standard sample, the standard sample including two or more analytes, each having a known quantity, the spectral data of the standard sample including a peak for each of the two or more analytes, and determining a bias parameter for each of the two or more analytes based on a peak for each of the two or more analytes of the standard sample.

In an example of the above aspect, the computer-implemented method further includes receiving, using the processor, a second data set including an spectral data of a sample of interest including the two or more analytes, the spectral data of the sample of interest including a peak for each of the two or more analytes, and determining one or more quantities of the two or more analytes in the sample of interest using the bias parameters for each of the two or more analytes.

### BRIEF DESCRIPTION OF THE DRAWINGS

FIGS. 1A and 1B are schematic views of an exemplary capillary electrophoresis system, in in accordance with various examples of the disclosure.

FIGS. 2 and 3 depict examples of fluorescence emission data, in accordance with various examples of the disclosure.

FIG. 4 is a table with data and calculated bias parameters, in accordance with various examples of the disclosure.

FIG. 5 is a flow chart illustrating a method of correcting detection bias, in accordance with various examples of the disclosure.

FIG. 6 is a schematic diagram illustrating one particular example of the computing device in accordance with various aspects and examples of the present disclosure.

### DETAILED DESCRIPTION

In some embodiments of this disclosure, methods, techniques, and systems are provided for detecting and/or correcting detection bias. In some embodiments, detection and/or correction of detection bias may provide certain advantages. For example, in some embodiments, by correcting a detection bias, a more accurate quantitation of analytes of a sample may be achieved (as compared to when no correction of a detection bias is performed). In some embodiments, detection and/or correction of bias may enable determination of conditions (e.g., conditions related to labeling and/or separation of analytes) under which detection bias is reduced or eliminated. For example, in some embodiments, when designing and/or optimizing labeling and/or separation methods, it could be helpful to compare detection bias between different conditions. Further, in some embodiments, where detection bias cannot be completely removed by method optimization, correcting for detection bias might be useful to achieve accurate quantification of analytes.

In some embodiments, methods for correcting detection bias are provided. In some embodiments, methods for correcting detection bias include detecting emission or absorbance data of a standard sample that includes two or more analytes, each having a known quantity. In some embodiments, the emission or absorbance data of the standard sample includes a peak for each of the two or more analytes. In some embodiments, methods for correcting detection bias further include determining a bias parameter for each of the two or more analytes based on the peak for each of the two or more analytes of the standard sample. In some embodiments, the methods may further include detecting emission or absorbance data of a sample of interest. In some embodiments, the sample of interest includes the two or more analytes. In some embodiments, the quantities of the two or more analytes are unknown. In some embodiments, the emission or absorbance data of the sample of interest included a peak for each of the two or more analytes. In some embodiments, the methods further comprise determining one or more quantities of the two or more analytes in the sample of interest using the bias parameters for each of the two or more analytes.

In some embodiments, determining the bias parameter for each of the two or more analytes comprises determining a peak area for each of the two or more analytes in the emission or absorbance data of the standard sample.

In some embodiments, quantities of analytes in a sample (e.g., standard sample and/or sample of interest) may be expressed using the following equation:

$${C}_{i} = {A}_{i} {f}_{i} / ( {\sum }_{k = 1}^{n} {A}_{k} {f}_{k} ) , \qquad ( 1 )$$

where n is the total number analytes in a sample,
i is an analyte index from 1 to n,
Cᵢ is the quantity of an i^{th} analyte in the sample,
Aᵢ is a peak area for the i^{th} analyte in the emission or absorbance data of the sample, and
fᵢ is the bias parameter for the i^{th} analyte.

In some embodiments, the peak area is the corrected peak area. In some embodiments, the corrected peak area in an electropherogram, obtained using, e.g., capillary electrophoresis, may be used. In some embodiments, Aᵢ is a peak area for the i^{th} analyte in the emission or absorbance data of the sample.

In some embodiments, determining the bias parameters is performed by using Equation (1), wherein n is a total number of the two or more analytes in the standard sample; i is an analyte index from 1 to n; Cᵢ is the known quantity of an i^{th} analyte in the standard sample; Aᵢ is the peak area for the i^{th} analyte in the emission or absorbance data of the standard sample; and fᵢ is the bias parameter for the i^{th} analyte. In some embodiments, determining one or more quantities of the two or more analytes in the sample of interest using the bias parameters for each of the two or more analytes.

In some embodiments, a sample (e.g., a standard sample and/or a sample of interest) includes the two or more analytes including a supercoiled (S), a linear (L), and/or a nicked-open circular (N) plasmids. In some embodiments, the quantities (e.g., concentrations) for the supercoiled (S), linear (L), and/or nicked-open circular (N) plasmids in a sample (e.g., a standard sample and/or a sample of interest) may be expressed using the following equations:

$${C}_{S} = {A}_{S} {f}_{S} / ( {A}_{S} {f}_{S} + {A}_{N} {f}_{N} + {A}_{L} {f}_{L} ) \qquad ( 2 )$$

$${C}_{L} = {A}_{L} {f}_{L} / ( {A}_{S} {f}_{S} + {A}_{N} {f}_{N} + {A}_{L} {f}_{L} ) \qquad ( 3 )$$

$${C}_{N} = {A}_{N} {f}_{N} / ( {A}_{S} {f}_{S} + {A}_{N} {f}_{N} + {A}_{L} {f}_{L} ) \qquad ( 4 )$$

wherein A_{S}, A_{N}, and A_{L} are the peak areas for the supercoiled (S), nicked/open circular (N), and linear (L) plasmids in the emission or absorbance data of the sample; C_{S}, C_{N}, and C_{L} are the quantities (e.g., concentrations) for the supercoiled (S), nicked/open circular (N), and linear (L) plasmids in the standard sample; and f_{S}, f_{N}, and f_{L} are the bias parameters for the supercoiled (S), nicked/open circular (N), and linear (L) plasmids. In some embodiments, the peak area is a corrected peak area. In some embodiments, the corrected peak area in an electropherogram, obtained using, e.g., capillary electrophoresis, may be used. In some embodiments, A_{S}, A_{N}, and A_{L} are the corrected peak areas for the supercoiled (S), nicked/open circular (N), and linear (L) plasmids in the emission or absorbance data of the sample.

In some embodiments, determining the bias parameters is performed by using Equations (2)-(4), wherein A_{S}, A_{N}, and A_{L} are the peak areas for the supercoiled (S), nicked/open circular (N), and linear (L) plasmids in the emission or absorbance data of the standard sample; C_{S}, C_{N}, and C_{L} are the known quantities for the supercoiled (S), nicked/open circular (N), and linear (L) plasmids in the standard sample; and f_{S}, f_{N}, and f_{L} are the bias parameters for the supercoiled (S), nicked/open circular (N), and linear (L) plasmids.

In some embodiments Equations (2)-(4) may be rewritten in the following form and used to determine the bias parameters:

$$\begin{matrix}{A}_{S} {f}_{S} = {C}_{S} ( {A}_{S} {f}_{S} + {A}_{N} {f}_{N} + {A}_{L} {f}_{L} ) \\ {A}_{N} {f}_{N} = {C}_{N} ( {A}_{S} {f}_{S} + {A}_{N} {f}_{N} + {A}_{L} {f}_{L} ) \\ {A}_{L} {f}_{L} = {C}_{L} ( {A}_{S} {f}_{S} + {A}_{N} {f}_{N} + {A}_{L} {f}_{L} )\end{matrix} ,$$

In some embodiments, determining the bias parameter for each of the two or more analytes includes determining an eigenvector (f) of a matrix having a formula A⁻¹ ca, wherein a is a row vector of the peak areas for the two or more analytes, c is a column vector of the known quantities of the two or more analytes of the standard sample, A is a diagonal matrix with the peak areas for the two or more analytes along the main diagonal, and wherein f is a column vector of the bias parameters for the two or more analytes. In some embodiments, the two or more analytes include a supercoiled (S), a linear (L), and a nicked/open circular (N) plasmids; and

$$a = ( {A}_{S} {A}_{N} {A}_{L} ) , \qquad ( 5 )$$

$$c = ( \begin{matrix}{C}_{S} \\ {C}_{N} \\ {C}_{L}\end{matrix} ) , \qquad ( 6 )$$

$${A}^{- 1} = ( \begin{matrix}1 / {A}_{S} & 0 & 0 \\ 0 & 1 / {A}_{N} & 0 \\ 0 & 0 & 1 / {A}_{L}\end{matrix} ) and \qquad ( 7 )$$

$$f = ( \begin{matrix}{f}_{S} \\ {f}_{N} \\ {f}_{L}\end{matrix} ) \qquad ( 8 )$$

wherein A_{S}, A_{N}, and A_{L} are the peak areas for the supercoiled (S), nicked/open circular (N), and linear (L) plasmids; wherein C_{S}, C_{N}, and C_{L} are the known quantities for the supercoiled (S), nicked/open circular (N), and linear (L) plasmids; and wherein f_{S}, f_{N}, and f_{L} are the bias parameters for the supercoiled (S), nicked/open circular (N), and linear (L) plasmids.

In some embodiments, determining the one or more quantities of the two or more analytes in the sample of interest using the bias parameters for each of the two or more analytes includes using equation:

$${}^{U} {C}_{i} = {}^{U} {A}_{i} {f}_{i} / ( {\sum }_{k = 1}^{n} {}^{U} {A}_{k} {f}_{k} ) \qquad ( 9 )$$

wherein n is a total number of the two or more analytes in the sample of interest; i is an analyte index from 1 to n; ^{U}Cᵢ is the quantity of an i^{th} analyte in the sample of interest; ^{U}Aᵢ is a peak area for the i^{th} analyte in the emission or absorbance data of the sample of interest; and f_{z} is the bias parameter for the i^{th} analyte. In some embodiments, Equation (9) is based on Equation (1) above.

### Example Capillary Electrophoresis Systems

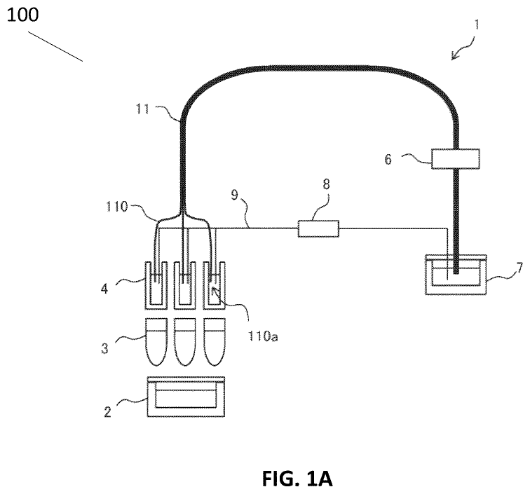

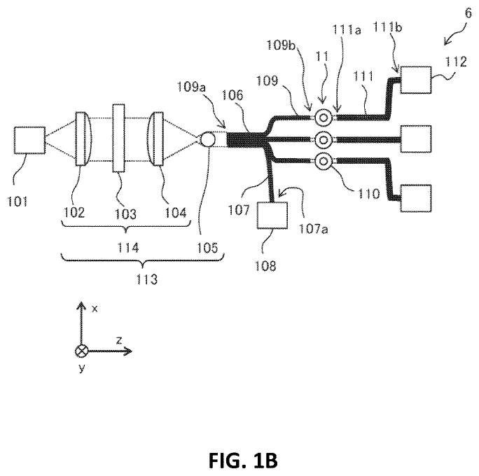

While other separation techniques may be used in some embodiments of this disclosure, these particular examples describe certain embodiments where electrophoretic separation is used. FIGS. 1A and 1B are schematic views of a capillary electrophoresis system that may be utilized to perform the methods in accordance with various examples of the disclosure. FIG. 1A is a schematic diagram illustrating a configuration example of a capillary electrophoresis device 1, and FIG. 1B is a schematic diagram illustrating a configuration example of detection unit 6 of the capillary electrophoresis device 1. As illustrated in FIG. 1A, the capillary electrophoresis device 1 may include a plurality of capillaries 110 into which samples to be measured are introduced and separated. In some embodiments, the electrophoresis device may include only one capillary. In some embodiments, instead of the capillaries illustrated in FIG. 1A, electrophoretic device may include one or more microfluidic channels in a chip. In an example in FIG. 1A, the plurality of capillaries 110 are bundled into a capillary array 11 at a position depicted in FIG. 1A (but such bundling may be done at other alternative position).

As illustrated in FIG. 1B, the detection unit 6 of the capillary electrophoresis device 1 may include a light source 101 (see FIG. 1B) that generates light for analyzing the samples separated (e.g., electrophoretically) in the plurality of capillaries 110. The detection unit of the capillary electrophoresis device 1 may further include detectors 112 (see FIG. 1B) that may detect light transmitted through the capillaries 110. In some embodiments, the electrophoresis device may comprise optical systems described in, e.g., WO 2021/095006, which is hereby incorporated by reference. In some examples, emission data of a sample may be detected. In some examples, absorbance data of a sample may be detected.

As illustrated in FIG. 1A, the capillary electrophoresis device 1 may include an electrophoretic medium container 2 accommodating an electrophoretic medium, and a plurality of sample containers 3 accommodating the samples to be analyzed. In the capillary electrophoresis device 1, the capillaries 110 may be connected to these containers, and the electrophoretic medium and/or the sample may be injected by electrical means, pressure, or the like. The capillary electrophoresis device 1 includes a plurality of injection-side electrode baths 4 and one discharge-side electrode bath 7. In some embodiments, a plurality of discharge-side electrode baths may be used. Each of the plurality of injection-side electrode baths 4 and the one discharge-side electrode bath 7 is filled with a buffer solution, and the capillaries 110 and an electrode 9 are immersed into their respective baths during electrophoretic separation. When a high-voltage power supply 8 applies a voltage, molecules in the samples move in the capillaries 110 from the injection side toward the discharge side while being separated according to properties such as a molecular weight and the amount of charge by the electrophoresis. Each of the molecules having moved is detected by optical means when reaching the detection unit 6.

As illustrated in FIG. 1B, the capillary electrophoresis device 1 includes an optical coupling optical system 113 and a plurality of irradiation optical fibers 109 between the light source 101 and the plurality of capillaries 110. The capillary electrophoresis device 1 includes a plurality of detection optical fibers 111 between the plurality of capillaries 110 and the detectors 112. In each of the plurality of irradiation optical fibers 109, one end face 109a is connected to the optical coupling optical system 113, and the other end face 109b is arranged close to the corresponding capillary 110. In each of the plurality of detection optical fibers 111, one end face 111a is arranged close to the corresponding capillary 110, and the other end face 111b is connected to the detector 112.

As further illustrated in FIG. 1B, the detection unit 6 of the capillary electrophoresis device 1 includes the optical coupling optical system 113 and the plurality of detectors 112. The optical coupling optical system 113 couples light from the light source 101 to the plurality of irradiation optical fibers 109. The optical coupling optical system 113 includes, for example, an imaging optical system 114 and at least one lens 105. The imaging optical system 114 in the present embodiment includes, for example, a lens 102 and a lens 104. The imaging optical system 114 may include a band-pass filter 103 between the lens 102 and the lens 104. That is, examples of a configuration of the optical coupling optical system 113 include the formation of the lens 102, the band-pass filter 103, the lens 104, and the lens 105. The light transmitted through the band-pass filter 103 is coupled to an optical fiber bundle (bundle 106) obtained by bundling the plurality of irradiation optical fibers 109 by the lens 104 and the lens 105. As a result, the light is split into the number of the irradiation optical fibers 109 included in the bundle 106. In an example, at least one optical fiber among the plurality of irradiation optical fibers 109 preferably has one end face 109a connected to the optical coupling optical system 113 and the other end face 107a connected to a detector 108 for reference light as illustrated in FIG. 1B.

### Illustrative Example: Sample Plasmids

While technologies and methods described herein may be implemented in any type of samples (e.g., protein samples), this particular example illustrate samples comprising plasmids. In a particular example, samples (such as those comprising DNA plasmids) are labeled using a fluorescent dye in order to facilitate detection when samples are analyzed (e.g., via capillary electrophoresis). Plasmids may exist in conformations including: supercoiled (S), linear (L), and nicked (N) (also known as open circular). Plasmid confirmation may affect their activity and/or suitability of plasmids for particular uses. Thus, there is a need to determine quantities of plasmid conformations present in a sample. For example, linear or nicked plasmids may be considered to be impurities in a supercoiled plasmid preparation and thus it may be beneficial to quantify their levels. In another example, supercoiled and nicked/open circular conformations might be considered impurities in a linear plasmid preparation and thus it may be beneficial to quantify their levels. In an example, a sample might comprise 3 different conformations of a single plasmid. These plasmid conformations may be separated by capillary electrophoresis and detected as, e.g., three different peaks corresponding to the supercoiled (S), linear (L), and nicked/open circular (N) forms. Ideally, the peak areas (which may be corrected for mobility) for each plasmid conformation would be representative of their quantity.

However, detection biases might be present. For example, the analysis of plasmid samples may be performed by introducing a fluorescent dye which associates with the DNA in such a manner that the molar emissivity thereof is increased by that association. The degree of fluorescence might be dependent on the degree to which the dye's association with DNA enhances its emission. The enhancement might vary with the environment in which the dye is found after its association with the DNA. For example. intercalating (e.g., intercalating dye) within the DNA molecule can act to prevent intramolecular rotations that quench fluorescence, but not all DNA conformations do this to the same extent. Further, as an example, the amount of dye associated with a plasmid can also vary depending on the affinity of the dye for the particular conformation of the DNA. These labeling effects result in peak areas that are greater or lesser than the actual relative abundance of the corresponding form of DNA in the sample. Thus, correction of detecting biases might be needed. For example, correcting of detection biases may include determining bias parameters for the analytes (e.g., plasmids).

In some embodiments, the quantities (e.g., concentrations) for each of the plasmid forms (Cₓ, where x is one of S, N or L) may be expressed according to Equations (2)-(4), based upon Equation (1).

If all bias parameters are each equal to unity, then no bias exists and the relative abundances are given by Equations (10)-(12):

$${C}_{S} = {A}_{S} / ( {A}_{S} + {A}_{N} + {A}_{L} ) \qquad ( 10 )$$

$${C}_{L} = {A}_{L} / ( {A}_{S} + {A}_{N} + {A}_{L} ) \qquad ( 11 )$$

$${C}_{N} = {A}_{N} / ( {A}_{S} + {A}_{N} + {A}_{L} ) \qquad ( 12 )$$

However, in some embodiments, the bias parameters may be greater than 1 or less than 1, in which case they may be used to correct any underestimation or overestimation of the relative abundance of DNA forms in the sample, respectively.

In some embodiments, correction of a detection bias includes detecting spectral data, such as spectrophotometric data, of a standard sample. In at least some embodiments, the spectral data is emission (e.g., spectrofluorometric) or absorbance data. In some embodiments, the standard sample comprises two or more analytes, each having a known quantity. In some embodiments, the emission or absorbance data of the standard sample comprises a peak for each of the two or more analytes. In some examples, correction of a detection bias includes determining a bias parameter for each of the two or more analytes based on the peak for each of the two or more analytes of the standard sample. For example, a standard sample may include supercoiled (S), a linear (L), and a nicked-open circular (N) plasmids, each having a known quantity. And, for example, the emission or absorbance data may include a peak for supercoiled (S), linear (L), and nicked/open circular (N) forms. For example, a peak for a supercoiled plasmid may have a peak area A_{S}; a peak for a linear plasmid may have a peak area A_{L}; a peak for a nicked plasmid may have a peak area A_{N}. In some embodiments, bias parameters f_{S}, f_{N}, and f_{L} (for the supercoiled, the nicked/open circular, and the linear plasmids) then can be determined using Equation (1). In some embodiments, these bias parameters are determined using Equations (2)-(4). For example, using the known quantities (C_{S}, C_{N}, C_{L}) and peak areas (A_{S}, A_{N}, and A_{L}) for the supercoiled, the nicked/open circular, and the linear plasmids of the standard sample, respectively, and Equation (1) or Equations (2)-(4), bias parameters f_{S}, f_{N}, and f_{L} can be determined. In some embodiments, the matrix-based method described above may be used, the matrix-based method including determining an eigenvector (f) of a matrix having a formula A⁻¹ca, wherein a is a row vector of the peak areas for the two or more analytes, c is a column vector of the known quantities of the two or more analytes of the standard sample, A is a diagonal matrix with the peak areas for the two or more analytes along the main diagonal, and wherein f is a column vector of the bias parameters for the two or more analytes. Equations (5)-(8) illustrate this method.

In some embodiments, correction of a detection bias further includes detecting emission or absorbance data of a sample of interest comprising the two or more analytes. In some embodiments, the emission or absorbance data of the sample of interest includes a peak for each of the two or more analytes. In some embodiments, correction of a detection bias further includes determining one or more quantities of the two or more analytes in the sample of interest using the bias parameters for each of the two or more analytes. For example, a sample of interest includes supercoiled (S), a linear (L), and/or a nicked-open circular (N) plasmids. And, for example, the emission or absorbance data for the sample of interest includes a peak for supercoiled (S), linear (L), and/or nicked/open circular (N) forms. For example, a peak for a supercoiled plasmid has a peak area A_{S}; a peak for a linear plasmid has a peak area A_{L}; a peak for a nicked plasmid has a peak area A_{N}. In some embodiments, bias parameters f_{S}, f_{N}, and f_{L} (for the supercoiled, the nicked/open circular, and the linear plasmids) is determined based on the peaks for each of the plasmids in the standard sample) and are used to determine one or more quantities of the supercoiled, a linear, and/or a nicked-open circular plasmids in the sample of interest. In some embodiments, Equation (9) is used to determine one or more quantities of the supercoiled, a linear, and/or a nicked-open circular plasmids in the sample of interest. In some embodiments, Equations (2)-(4) are used to determine one or more quantities of the supercoiled, a linear, and/or a nicked-open circular plasmids in the sample of interest. For example, using peak areas (A_{S}, A_{N}, and A_{L}) and bias parameters (f_{S}, f_{N}, and f_{L}) for the supercoiled, the nicked/open circular, and the linear plasmids of the sample of interest, respectively, and Equation (9) or Equations (2)-(4), quantities (C_{S}, C_{N}, C_{L}) for the sample of interest can be determined.

Although three (3) peaks (for the supercoiled, linear, and nicked/open circular plasmids) are illustrated herein and discussed throughout this disclosure, other numbers of peaks may appear in the emission or absorbance data of a sample (e.g., a standard sample and/or a sample of interest). For example, a fourth peak may appear that may correspond to a dimer where the dimer may be caused by the association of two or more plasmids to form a complex that migrates differently from the singleton plasmids. In some examples, a sample may comprise two analytes; it's emission and absorbance data may comprise a peak for each of the two analytes (e.g., two peaks). For example, some samples may have nearly no linear form or nearly no open circular form. Accordingly, the examples of the disclosure may be used to resolve bias in more than three (3) peaks, or in less than three (3) peaks, of the fluorescence emission data. The equations discussed above may be adjusted to reflect the actual number of peaks, whether less than three (3) or more than three (3).

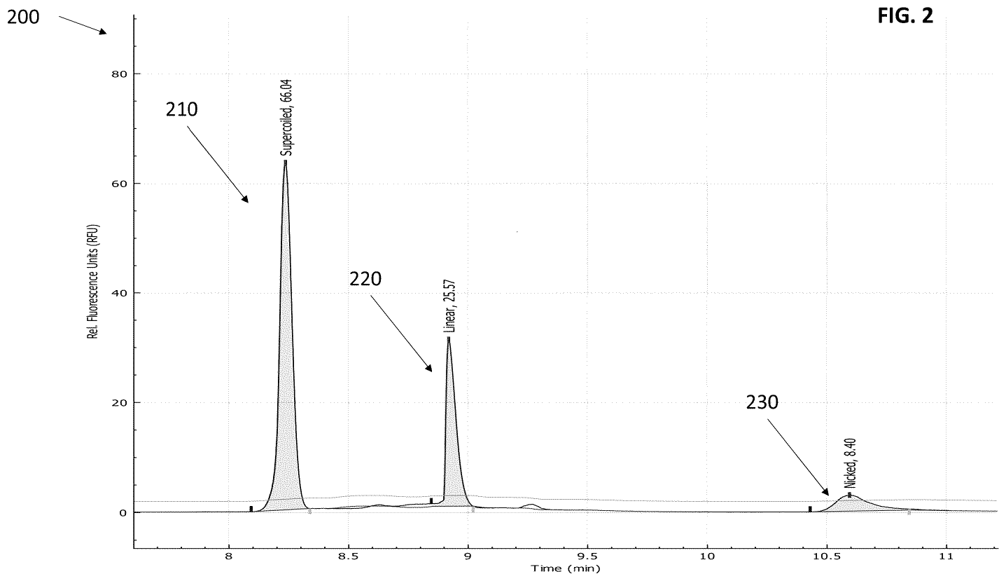

FIG. 2 illustrates an example of a fluorescence emission data of a sample (comprising plasmids having known quantities), in accordance with various examples of the disclosure. In FIG. 2, the emission data (e.g., electropherogram 200) was collected for a standard sample comprising a 1:1 mixture of two plasmids, referred to respectively as RFI and RFII, where RFI was 90% supercoiled, and RFII was 90% nicked/open circular. Accordingly, in this example, the linear form of the plasmid is considered to be an impurity. The example of the fluorescence emission data 200 shows a peak 210 (for the supercoiled configuration), peak 220 (for the linear configuration), and peak 230 (for the nicked/open circular configuration). The peak area of each configuration may be determined based on the fluorescence emission data 200. With respect to FIG. 2, the known concentration of the supercoiled plasmid configuration C_{S}, of the nicked/open circular plasmid configuration C_{N}, and of the linear plasmid configuration C_{L} are 0.475, 0.475, and 0.05, respectively. Referring back to FIG. 2, integration of the peaks 210, 220 and 230 yields the following values for peak areas A_{S}, A_{N}, and A_{L} as being equal to 66.04, 8.40, and 25.57, respectively. Accordingly, by relying on Equations (2)-(4) discussed above, it is possible to calculate the bias parameters and f_{S}, f_{N}, and f_{L} as being equal to 0.1261, 0.9914, and 0.0343, respectively. In another example, biasing parameters may be determined using the matrix method described above with respect to Equations (5)-(8).

Although peaks 210, 220, and 230 are illustrated in an ordered manner, where the supercoiled peak 210 corresponds to the shortest time, followed by the linear peak 220, and then followed by the nicked/open circular peak 230, the peaks may be in any order. In one example, the nicked/open circular peak 230 may appear first, flowed by the supercoiled peak 210, and then the linear peak 220. Other orders of appearance of the various peaks 210, 220 and 230 may also occur.

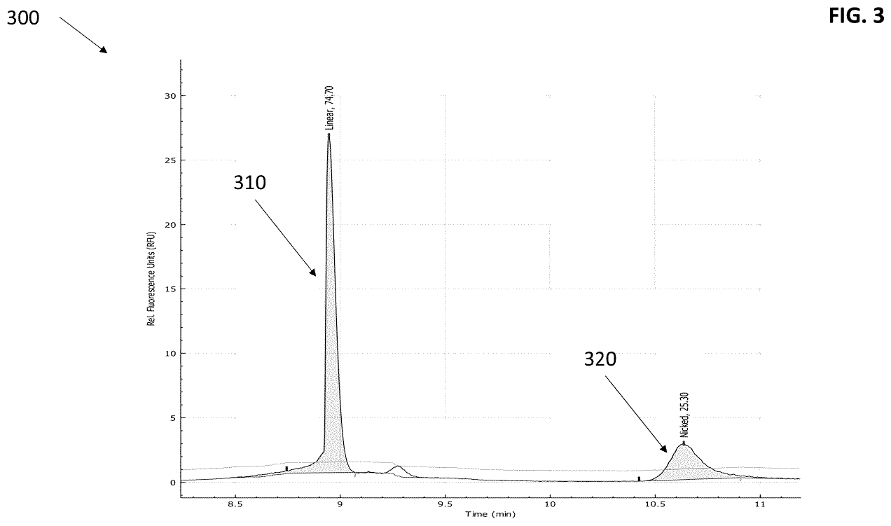

FIG. 3 illustrates an example of fluorescence emission data for a sample of interest (comprising linear and nicked-open circular plasmids), in accordance with various examples of the disclosure. In this example, the quantities (e.g., concentration) of each plasmid are assumed to be unknown for illustrative purposes. The peak areas for peaks 320 and 310 (A_{N} and A_{L}) are 25.30 and 74.7, respectively. Using the bias parameters f_{S}, f_{N}, and f_{L} determined based on the data illustrated in FIG. 2 and using Equations (2)-(4) above, the concentrations of each plasmid in the sample of interest can be determined. Accordingly, C_{S}, C_{N}, and C_{L} in the sample of interest in this example were determined to be 0, 0.907, and 0.093, respectively. In this example, these values are in alignment with the 90% of the nicked plasmid value determined for this sample of interest using a different method. In this example, while the peak area for the linear was the greatest, after the bias correction was performed, it was determined that the linear plasmid was present in the sample of interest in a smaller quantity as compared to the nicked form (as C_{N} was 0.907 and C_{L} in was 0.093). It should be noted that in the spectrum illustrated in FIG. 3, there is no peak corresponding to the supercoiled configuration. Hence, in Equations (2)-(4), As and C_{S} are equal to zero. So three-variable Equations (2)-(4) are simplified into two-variable equations.

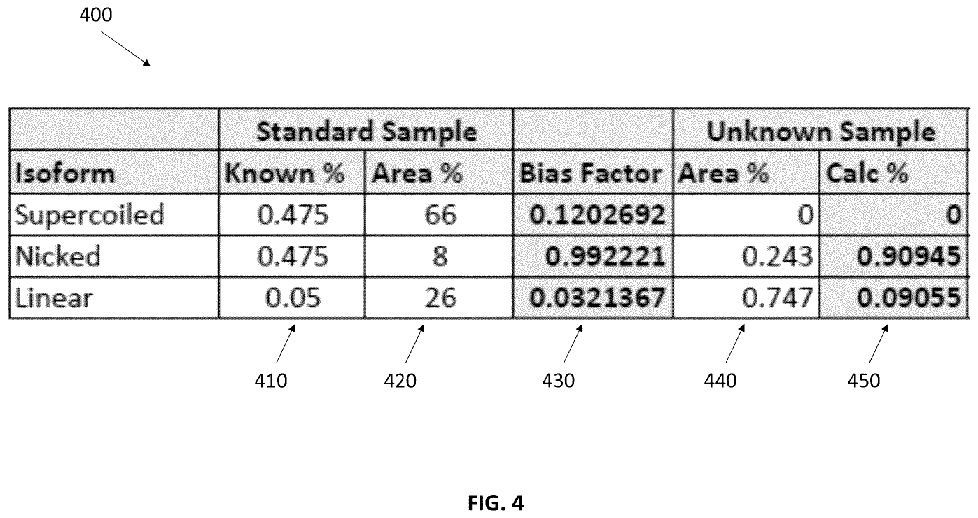

FIG. 4 is a table illustrating determined bias parameters, in accordance with various examples of the disclosure. In Table 400 illustrated in FIG. 4, columns 410 and 420 illustrate a standard sample (comprising analytes with known quantities) and columns 440 and 450 illustrate a sample of interest (labeled as “unknown sample,” comprising analytes quantities of which were determined). For each sample and for each plasmid configuration of each sample, e.g., supercoiled, nicked/open circular, and linear, the quantities (known in column 410 or calculated % in column 450) and the peak areas in columns 420 and 440 are indicated. As discussed above with respect to FIG. 2, based on the known quantities in column 410 and the peak areas in column 420, the biasing parameters f_{S}, f_{N}, and f_{L} may be calculated based on Equations (2)-(4) discussed above and indicated in column 430. Based on the biasing parameters indicated in column 430 and based on the peak areas determined from the data of a sample of interest indicated in column 440, it then becomes possible, based on the above Equations (2)-(4), to determine the quantities of each plasmid configuration (e.g., a concentration), e.g., of the supercoiled configuration, of the nicked/open circular configuration, and of the linear configuration, in column 450.

In some embodiments, a computer-implemented method for correcting detection bias includes receiving, using a processor, a first data set including an emission or absorbance data of a standard sample, the standard sample including two or more analytes, each having a known quantity, the emission or absorbance data of the standard sample including a peak for each of the two or more analytes; determining a bias parameter for each of the two or more analytes based on a peak for each of the two or more analytes of the standard sample. In some embodiments, the computer-implemented method further includes receiving, using the processor, a second data set including an emission or absorbance data of a sample of interest including the two or more analytes, the emission or absorbance data of the sample of interest comprising a peak for each of the two or more analytes; and determining one or more quantities of the two or more analytes in the sample of interest using the bias parameters for each of the two or more analytes. In some embodiments, a computer-implemented method further includes generating a visual representation of known quantities for the two or more analytes of the standard sample, peak areas for the two or more analytes of the standard sample, bias parameters, peak areas for the two or more analytes of the sample of interest, and/or the quantities for the two or more analytes of the sample of interest. For example, the computer-implemented method may generate a table like or similar to the one illustrated in FIG. 4.

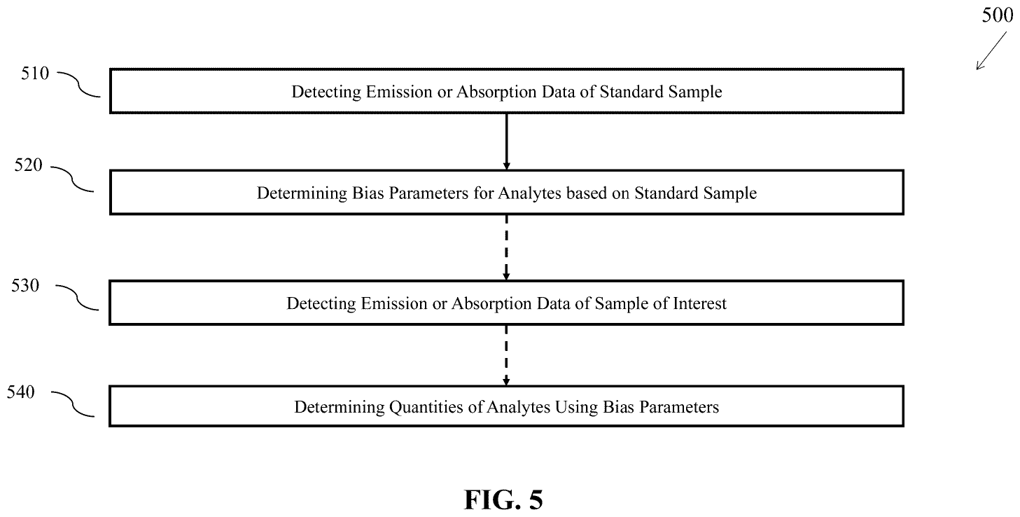

FIG. 5 is a flow chart illustrating a method of correcting detection bias in fluorescence data, in accordance with various examples of the disclosure. In examples, the method 500 includes operation 510, which includes detecting emission or absorbance data of a standard sample that has two or more analytes, each having a known quantity. The emission or absorbance data of the standard sample may include a peak for each of the two or more analytes. The standard sample may be, e.g., a plasmid, and the two or more analytes may include different forms of the plasmid (e.g., a supercoiled form, a linear form, and a nicked/open circular form). In some embodiments, the two or more analytes may include a protein or a nucleic acid. The standard sample may be subjected to capillary electrophoresis separation in a separation matrix prior to detecting the emission or absorbance data. The standard sample may be labeled with a dye prior to detecting the emission or absorbance data.

With reference to FIGS. 2 and 4, in some embodiments, this operation 510 may correspond to collecting an electropherogram of a standard (comprising two or more analytes, each having a known quantity). For example, with respect to FIG. 4, the known quantities are illustrated in column 410. In some embodiments, such electropherogram may include the supercoiled peak 210, the linear peak 220, and the nicked/open circular peak 230. In some examples, the areas under each peak may be determined—for example, as indicated in column 420 of table 400 in FIG. 4.

In some embodiments, operation 520 includes determining a bias parameter for each of the two or more analytes based on the peak for each of the two or more analytes of the standard sample. In some embodiments, operation 520 includes determining bias parameters for the known plasmid. For example, this operation 520 may be accomplished by relying on Equations (2)-(4) discussed above while knowing the values of C_{N}, C_{L} and C_{S}, and the values of A_{N}, A_{L} and A_{S}, to derive the biasing parameters f_{N}, f_{L} and f_{S}. For example, with reference to table 400 in FIG. 4, this operation 520 results in the determination of the biasing parameters indicated in column 430. Determining the bias parameter for each the two or more analytes may include determining a peak area for each of the two or more analytes in the emission or absorbance data of the standard sample. As noted above in the context of Equation (1), determining the bias parameters may be performed using the following equation: Ci=Aᵢfᵢ/(Σₖ₌₁ⁿ Aₖfₖ), where n is a total number of the two or more analytes in the standard sample, i is an analyte index from 1 to n, Cᵢ is the known quantity of an i^{th} analyte in the standard sample, Aᵢ is the peak area for the it analyte in the emission or absorbance data of the standard sample, and fᵢ is the bias parameter for the i^{th} analyte.

In some embodiments, the number of analytes n is equal to 3 for the supercoiled form of a plasmid, the linear form of the plasmid, and the nicked/open circular form of the plasmid. Accordingly, determining the bias parameters may be performed by using equations (1)-(3) above where A_{S}, A_{N}, and A_{L} are the peak areas for the supercoiled (S), nicked/open circular (N), and linear (L) plasmids in the emission or absorbance data of the standard sample, C_{S}, C_{N}, and C_{L} are the known quantities for the supercoiled (S), nicked/open circular (N), and linear (L) plasmids in the standard sample, and f_{S}, f_{N}, and f_{L} are the bias parameters for the supercoiled (S), nicked/open circular (N), and linear (L) plasmids.

In other examples, determining the bias parameter for each the two or more analytes comprises determining an eigenvector (f) of a matrix having a formula A⁻¹ca, where “a” is a row vector of the peak areas for the two or more analytes, “c” is a column vector of the known quantities of the two or more analytes of the standard sample, “A” is a diagonal matrix with the peak areas for the two or more analytes along the main diagonal, and “f” is a column vector of the bias parameters for the two or more analytes. In the case the analytes includes plasmids having a supercoiled form (S), a linear form (L) and a nicked/open circular form (N), “a,” “c,” “A,” and “f” may be respectively defined as in Equations (5)-(8) above.

In equations (5)-(8) above, A_{S}, A_{N}, and A_{L} are the peak areas for the supercoiled (S), nicked/open circular (N), and linear (L) plasmids, C_{S}, C_{N}, and C_{L} are the known quantities for the supercoiled (S), nicked/open circular (N), and linear (L) plasmids, and f_{S}, f_{N}, and f_{L} are the bias parameters for the supercoiled (S), nicked/open circular (N), and linear (L) plasmids.

In some embodiments, operation 520 may also include determining the bias parameters for each of the two or more analytes under two or more different conditions to determine under which of the two or more different conditions the bias parameters are closer to 1. Determining the two or more conditions may include, e.g., (a) different separation matrices used for capillary electrophoresis separation of the standard sample, and/or (b) different dyes used for labeling the standard sample.

In some embodiments, operation 530 includes detecting emission or absorbance data of a sample of interest that includes the two or more analytes, the emission or absorbance data of the sample of interest comprising a peak for each of the two or more analytes. For example, detecting the emission or absorbance data of the sample of interest may include detecting a fluorescence emission data of a sample of interest comprising a plasmid. For example, a sample of interest may comprise supercoiled, nicked or open circular, and/or linear plasmids in unknown quantities. However, in some embodiments, the biasing parameters are known from operation 520, and the peak areas may be determined based on the detected fluorescence emission data. For example, with reference to FIG. 3, the peak areas may be determined for peaks 310 and 320.

In some embodiments, operation 540 includes determining one or more quantities of the two or more analytes in the sample of interest using the bias parameters for each of the two or more analytes. For example, determining the quantities of the two or more analytes in the sample of interest may include determining one or more concentrations of the unknown plasmid based on the determined one or more bias parameters. For example, operation 540 includes determining a concentration of one or more of the plasmid configurations, e.g., supercoiled, nicked or open circular, or linear. In the examples of FIG. 3, the electropherogram 300 includes peaks for the linear configuration and the nicked or open circular configuration. Accordingly, based on Equations (3) and (4) above, it is possible to determine the respective concentrations of the linear configuration and the nicked or open circular configuration. In this case, Equation (2) is not necessary because no peak for the supercoiled configuration is illustrated in FIG. 3.

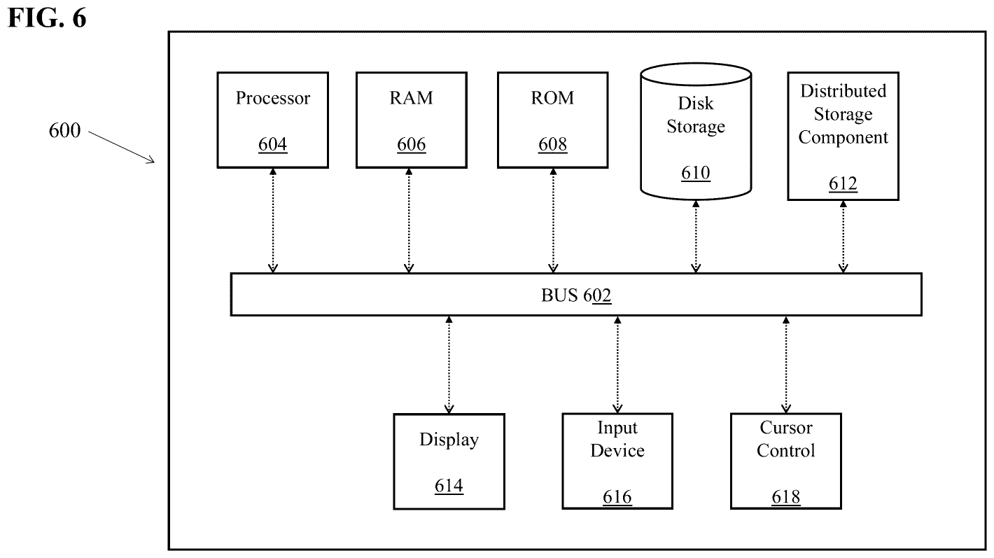

FIG. 6 is a schematic diagram illustrating one particular example of the computing device in accordance with various aspects and examples of the present disclosure. Now referring to FIG. 6, an example of the computing device 600 that may be used to control operation of the capillary electrophoresis system 100 illustrated in FIGS. 1A-1B, as illustrated and described. In the illustrated example of FIG. 6, the computing device 600 may include a bus 602 or other communication mechanism of similar function for communicating information, and at least one processing element 604 coupled with bus 602 for processing information. As is appreciated by those skilled in the relevant arts, such at least one processing element 604 may include a plurality of processing elements or cores, which may be packaged as a single processor or in a distributed arrangement. Furthermore, in some examples, a plurality of virtual processing elements 604 may be included in the computing device 600 to provide the control or management operations for the capillary electrophoresis system 100 illustrated in FIGS. 1A-1B, as well as to correct for detection bias, e.g., by detecting emission or absorbance data, determining a bias parameter, and other operations described herein.

Computing device 600 may also include one or more volatile memory(ies) 606, which can for example include random access memory(ies) (RAM) or other dynamic memory component(s), coupled to one or more busses 602 for use by the at least one processing element 604. Computing device 600 may further include static, non-volatile memory(ies) 608, such as read only memory (ROM) or other static memory components, coupled to busses 602 for storing information and instructions for use by the at least one processing element 604. A storage component 610, such as a storage disk or storage memory, may be provided for storing information and instructions for use by the at least one processing element 604. As is appreciated, in some examples the computing device 600 may include a distributed storage component 612, such as a networked disk or other storage resource available to the computing device 600.

Computing device 600 may be coupled to one or more displays 614 for displaying information to a computer user. Optional user input devices 616, such as a keyboard and/or touchscreen, may be coupled to a bus for communicating information and command selections to the at least one processing element 604. An optional graphical input device 618, such as a mouse, a trackball or cursor direction keys for communicating graphical user interface information and command selections to the at least one processing element. The computing device 600 may further include an input/output (I/O) component, such as a serial connection, digital connection, network connection, or other input/output component for allowing intercommunication with other computing components and the various components of the capillary electrophoresis system 100 illustrated in FIGS. 1A-1B.

In various examples, computing device 600 can be connected to one or more other computer systems a network to form a networked system. Such networks can for example include one or more private networks, or public networks such as the Internet. In the networked system, one or more computer systems can store and serve the data to other computer systems. The one or more computer systems that store and serve the data can be referred to as servers or the cloud, in a cloud computing scenario. The one or more computer systems can include one or more web servers, for example. The other computer systems that send and receive data to and from the servers or the cloud can be referred to as client or cloud devices, for example. Various operations of the capillary electrophoresis system 100 illustrated in FIGS. 1A-1B may be supported by operation of the distributed computing systems. Further, the processes and operations related to the correction of detection bias may also be performed within such distributed computing systems.

Computing device 600 may be operative to control operation of the components of the capillary electrophoresis system 100 illustrated in FIGS. 1A-1B and to handle data generated by components thereof. Such data may be used by the computing device to perform the various processes related to detection bias correction. In some examples, analysis results are provided by computing device 600 in response to the at least one processing element 604 executing instructions contained in memory 606 or 608 and performing operations on data received from the capillary electrophoresis system 100. Execution of instructions contained in memory 606 or 608 by the at least one processing element 604 can render the capillary electrophoresis system 100 operative to perform the various methods described herein. Alternatively, hard-wired circuitry may be used in place of or in combination with software instructions to implement the present teachings. Thus, implementations of the present teachings are not limited to any specific combination of hardware circuitry and software.

The term “computer-readable medium” as used herein refers to any media that participates in providing instructions to processor 604 for execution. Such a medium may take many forms, including but not limited to, non-volatile media, volatile media, and transmission media. Non-volatile media includes, for example, optical or magnetic disks, such as disk storage 610. Volatile media includes dynamic memory, such as memory 606. Transmission media includes coaxial cables, copper wire, and fiber optics, including the wires that include bus 602.

Common forms of computer-readable media or computer program products include, for example, a floppy disk, a flexible disk, hard disk, magnetic tape, or any other magnetic medium, a CD-ROM, digital video disc (DVD), a Blu-ray Disc, any other optical medium, a thumb drive, a memory card, a RAM, PROM, and EPROM, a FLASH-EPROM, any other memory chip or cartridge, or any other tangible medium from which a computer can read.

Various forms of computer readable media may be involved in carrying one or more sequences of one or more instructions to processor 604 for execution. For example, the instructions may initially be carried on the magnetic disk of a remote computer. The remote computer can load the instructions into its dynamic memory and send the instructions over a telephone line using a modem. A modem local to computer system 600 can receive the data on the telephone line and use an infra-red transmitter to convert the data to an infra-red signal. An infra-red detector coupled to bus 602 can receive the data carried in the infra-red signal and place the data on bus 602. Bus 602 carries the data to memory 606, from which processor 604 retrieves and executes the instructions. The instructions received by memory 606 may optionally be stored on storage device 610 either before or after execution by processor 604.

In accordance with various examples, instructions configured to be executed by a processor to perform a method are stored on a computer-readable medium. The computer-readable medium can be a device that stores digital information. For example, a computer-readable medium includes a compact disc read-only memory (CD-ROM) as is known in the art for storing software. The computer-readable medium is accessed by a processor suitable for executing instructions configured to be executed.

This disclosure described some examples of the present technology with reference to the accompanying drawings, in which only some of the possible examples were shown. Other aspects can, however, be embodied in many different forms and should not be construed as limited to the examples set forth herein. Rather, these examples were provided so that this disclosure was thorough and complete and fully conveyed the scope of the possible examples to those skilled in the art.

Although specific examples were described herein, the scope of the technology is not limited to those specific examples. One skilled in the art will recognize other examples or improvements that are within the scope of the present technology. Therefore, the specific structure, acts, or media are disclosed only as illustrative examples. Examples according to the technology may also combine elements or components of those that are disclosed in general but not expressly exemplified in combination, unless otherwise stated herein. The scope of the technology is defined by the following claims and any equivalents therein.

What is claimed is:

1. A method for correcting detection bias, the method comprising:
detecting spectral data of a standard sample, the standard sample comprising two or more analytes, each having a known quantity, the spectral data of the standard sample comprising a peak for each of the two or more analytes; and
determining a bias parameter for each of the two or more analytes based on the peak for each of the two or more analytes of the standard sample.

2. The method of claim 1, further comprising:
detecting spectral data of a sample of interest comprising the two or more analytes, the spectral data of the sample of interest comprising a peak for each of the two or more analytes; and
determining quantities of the two or more analytes in the sample of interest using the bias parameters for each of the two or more analytes.

3. The method of claim 2, wherein determining the quantities of the two or more analytes in the sample of interest using the bias parameters for each of the two or more analytes is performed according to:
$${}^{U} {C}_{i} = {}^{U} {A}_{i} {f}_{i} / ( {\sum }_{k = 1}^{n} {}^{U} {A}_{k} {f}_{k} ) ;$$
wherein:
n is a total number of the two or more analytes in the sample of interest;
i is an analyte index from 1 to n;
^{U}Cᵢ is the quantity of an i^{th} analyte in the sample of interest;
^{U}Aᵢ is a peak area for the i^{th} analyte in the spectral data of the sample of interest; and
fᵢ is the bias parameter for the i^{th} analyte.

4. The method of claim 1, wherein determining the bias parameter for each the two or more analytes comprises determining a peak area for each of the two or more analytes in the spectral data of the standard sample.

5. The method of claim 1, wherein determining the bias parameter for each of the two or more analytes is performed by using equation:
$$Ci = {A}_{i} {f}_{i} / ( {\sum }_{k = 1}^{n} {A}_{k} {f}_{k} ) ;$$
wherein:
n is a total number of the two or more analytes in the standard sample,
i is an analyte index from 1 to n,
Cᵢ is the known quantity of an i^{th} analyte in the standard sample,
Aᵢ is the peak area for the i^{th} analyte in the spectral data of the standard sample, and
fᵢ is the bias parameter for the i^{th} analyte.

6. The method of claim 1, wherein the two or more analytes comprise at least two of a supercoiled (S) plasmid, a linear (L) plasmid, and a nicked/open circular (N) plasmid.

7. The method of claim 6, wherein
the two or more analytes comprise the supercoiled (S) plasmid, the linear (L) plasmid, and the nicked-open circular (N) plasmid; and
determining the bias parameters is performed by using equations:
$$\begin{matrix}{A}_{S} {f}_{S} = {C}_{S} ( {A}_{S} {f}_{S} + {A}_{N} {f}_{N} + {A}_{L} {f}_{L} ) \\ {A}_{N} {f}_{N} = {C}_{N} ( {A}_{S} {f}_{S} + {A}_{N} {f}_{N} + {A}_{L} {f}_{L} ) \\ {A}_{L} {f}_{L} = {C}_{L} ( {A}_{S} {f}_{S} + {A}_{N} {f}_{N} + {A}_{L} {f}_{L} )\end{matrix} ,$$
wherein:
A_{S}, A_{N}, and A_{L} are the peak areas for the supercoiled (S) plasmid, nicked-open circular (N) plasmid, and linear (L) plasmid, respectively, in the spectral data of the standard sample,
C_{S}, C_{N}, and C_{L} are the known quantities for the supercoiled (S) plasmid, nicked/open circular (N) plasmid, and linear (L) plasmid, respectively, in the standard sample, and
f_{S}, f_{N}, and f_{L} are the bias parameters for the supercoiled (S) plasmid, nicked/open circular (N) plasmid, and linear (L) plasmid, respectively.

8. The method of claim 6, wherein:
the two or more analytes comprise the supercoiled (S) plasmid, the linear (L) plasmid, and the nicked/open circular (N) plasmid,
$$\begin{matrix}a = ( {A}_{S} {A}_{N} {A}_{L} ) , \\ c = ( \begin{matrix}{C}_{S} \\ {C}_{N} \\ {C}_{L}\end{matrix} ) , \\ {A}^{- 1} = ( \begin{matrix}1 / {A}_{S} & 0 & 0 \\ 0 & 1 / {A}_{N} & 0 \\ 0 & 0 & 1 / {A}_{L}\end{matrix} ) , and \\ f = ( \begin{matrix}{f}_{S} \\ {f}_{N} \\ {f}_{L}\end{matrix} ) ,\end{matrix}$$
wherein:
A_{S}, A_{N}, and A_{L} are the peak areas for the supercoiled (S) plasmid, the nicked/open circular (N) plasmid, and the linear (L) plasmid, respectively,
C_{S}, C_{N}, and C_{L} are the known quantities for the supercoiled (S) plasmid, the nicked/open circular (N) plasmid, and the linear (L) plasmid, respectively, and
f_{S}, f_{N}, and f_{L} are the bias parameters for the supercoiled (S) plasmid, the nicked/open circular (N) plasmid, and the linear (L) plasmid, respectively.

9. The method of claim 1, wherein determining the bias parameter for each the two or more analytes comprises determining an eigenvector (f) of a matrix having a formula A⁻¹ca, and wherein:
a is a row vector of the peak areas for the two or more analytes,
c is a column vector of the known quantities of the two or more analytes of the standard sample,
A is a diagonal matrix with the peak areas for the two or more analytes along the main diagonal, and
wherein f is a column vector of the bias parameters for the two or more analytes.

10. The method of claim 1, wherein the two or more analytes comprise at least one of a protein and a nucleic acid.

11. The method of claim 1, wherein the standard sample is subjected to capillary electrophoresis separation in a separation matrix prior to detecting the spectral data.

12. The method of claim 1, wherein the standard sample is labeled with a dye prior to detecting the spectral data.

13. The method of claim 1, further comprising determining the bias parameters for each of the two or more analytes under two or more different conditions to determine under which of the two or more different conditions the bias parameters are closer to 1.

14. The method of claim 13, wherein the two or more different conditions comprise at least one of:
different separation matrices used for capillary electrophoresis separation of the standard sample; and
different dyes used for labeling the standard sample.

15. The method of claim 1, wherein the spectral data is one of emission and absorbance data.

16. A computer-implemented method for correcting detection bias comprising:
receiving, using a processor, a first data set comprising spectral data of a standard sample, the standard sample comprising two or more analytes, each having a known quantity, the spectral data of the standard sample comprising a peak for each of the two or more analytes; and
determining a bias parameter for each of the two or more analytes based on a peak for each of the two or more analytes of the standard sample.

17. The computer-implemented method of claim 16, and further comprising:
receiving, using the processor, a second data set comprising spectral data of a sample of interest comprising the two or more analytes, the spectral data of the sample of interest comprising a peak for each of the two or more analytes; and
determining one or more quantities of the two or more analytes in the sample of interest using the bias parameters for each of the two or more analytes.

18. The computer-implemented method of claim 16, wherein the spectral data is one of absorbance and emission data.
